SEQUENCE LISTING (10 sequences)

SEQ ID NO: 1 - DNA, 22 bp, ARTIFICIAL
  PRIMER FOR PCR OF BARLEY OR CEREAL YELLOW DWARF VIRUS
tgttgaggagtctacctatttg

SEQ ID NO: 2 - DNA, 20 bp, ARTIFICIAL
  PRIMER FOR PCR OF BARLEY OR CEREAL YELLOW DWARF VIRUS
tacgctaagtgcccaactcc

SEQ ID NO: 3 - DNA, 24 bp, ARTIFICIAL
  PRIMER FOR PCR OF BARLEY OR CEREAL YELLOW DWARF VIRUS
tcaccttcgggccgtctctatcag

SEQ ID NO: 4 - DNA, 24 bp, ARTIFICIAL
  PRIMER FOR PCR OF BARLEY OR CEREAL YELLOW DWARF VIRUS
tcaccttcggggcgtctctttctg

SEQ ID NO: 5 - DNA, 20 bp, ARTIFICIAL
  PRIMER FOR PCR OF BARLEY OR CEREAL YELLOW DWARF VIRUS
acctagacgcgcaaatcaaa

SEQ ID NO: 6 - DNA, 20 bp, ARTIFICIAL
  PRIMER FOR PCR OF BARLEY OR CEREAL YELLOW DWARF VIRUS
acatttcttcgtgtgttgcg

SEQ ID NO: 7 - DNA, 20 bp, ARTIFICIAL
  PRIMER FOR PCR OF BARLEY OR CEREAL YELLOW DWARF VIRUS
acatttttgcgtgcgttgcg

SEQ ID NO: 8 - DNA, 20 bp, ARTIFICIAL
  PRIMER FOR PCR OF BARLEY OR CEREAL YELLOW DWARF VIRUS
aataaccgcaggagaaatgg

SEQ ID NO: 9 - DNA, 15 bp, ARTIFICIAL
  PRIMER FOR PCR OF LUTEOVIRUS
ccagtggttrtggtc

SEQ ID NO: 10 - DNA, 14 bp, ARTIFICIAL
  PRIMER FOR PCR OF LUTEOVIRUS
gtctacctatttcc

(19) United States
(12) Patent Application Publication
(10) Pub. No.: US 2007/0065812 A1
(43) Pub. Date: Mar. 22, 2007

(54) Multiplexed RT-PCR assay for detection and separation of barley and cereal yellow dwarf viruses

(75) Inventors: Carolyn M. Malmstrom, East Lansing, MI (US); Ruijie Shu, East Lansing, MI (US)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: Michigan State University

(21) Appl. No.: 11/150,036

(22) Filed: Jun. 10, 2005

Related U.S. Application Data
(60) Provisional application No. 60/579,270, filed on Jun. 14, 2004.

(51) Int. Cl. C12Q 1/70; C12Q 1/68; C07H 21/04

(52) U.S. Cl. 435/5; 435/6; 536/24.1

(57) ABSTRACT

RT-PCR primer multiplexes and a method for detecting the presence of Subgroup I and II barley or cereal yellow dwarf viruses in a sample. A first multiplex includes a first primer pair that selectively amplifies Subgroup I viruses, and a second primer pair that selectively amplifies Subgroup II viruses. A second multiplex includes primer pairs that also selectively amplify Subgroup I BYDV-PAV, BYDV-MAV, and BYDV-SGV. The PCR fragments resulting from the multiplexes differ in size. Therefore, with the first multiplex, a sample can be identified as containing either a Subgroup I or a Subgroup II barley or cereal yellow dwarf virus, and with the second multiplex, specific Subgroup I virus in the sample also can be identified. The present invention also relates to a kit including these RT-PCR primer multiplexes.

### CROSS-REFERENCE TO RELATED APPLICATIONS

This application claims benefit of provisional application Ser. No. 60/579,270, filed Jun. 14, 2004, entitled MULTIPLEXED RT-PCR ASSAY FOR DETECTION AND SEPARATION OF BARLEY AND CEREAL YELLOW DWARF VIRUSES, the entire contents of which are incorporated herein in their entirety.

### GOVERNMENT INTERESTS

The U.S. Government has a paid-up license in the invention and the right in limited circumstances to require the patent owner to license others on reasonable terms as provided by the terms of Grant No. DEB 9983373 awarded by the National Science Foundation.

### FIELD OF THE INVENTION

This invention relates to methods for detecting yellow dwarf viruses in barley and cereal.

### BACKGROUND OF THE INVENTION

Virologists and ecologists are increasingly realizing that plant viruses may play broad roles in regulating the dynamics of natural vegetation and ecosystem function (Drewes Milligan and Cosper, 1994; Gilbert, 2002; Harper, 1977; Malmstrom, 1998; Nienhaus and Castello, 1989; Power and Remold, 1996; Wommack and Colwell, 2000). However, development of understanding of these roles has been held back by the cost and difficulty of accurately diagnosing viral infection in large numbers of natural hosts, as required for studies of natural population dynamics.

Barley and cereal yellow dwarf viruses are some of the most serious and widespread viruses of cereals worldwide. These viruses can infect barley, oats, rye, and wheat as well as numerous species of grasses. Thus, there is a need for a cost-effective and streamlined approach for molecular diagnosis of infection by the barley and cereal yellow dwarf viruses (B/CYDVs), which form a globally significant group of Luteoviridae found in cereals and natural grasslands (Irwin and Thresh, 1990). These single-stranded, positive-sense RNA viruses are currently classified as six related species: two luteoviruses (BYDV-PAV, BYDV-MAV), one polerovirus (CYDV-RPV), and three unassigned Luteoviridae (BYDV-RMV, BYDV-SGV and BYDV-GPV) (Barker and Smith 1999). The viruses are aphid-transmitted and the three-letter suffixes reflect initial assessments of vector specificity (Gray and Gildow, 2003), which are now understood to be more variable than first appeared (Creamer and Falk, 1989; Lister and Rochow, 1988; Plumb, 1974; Yount and Carroll, 1983).

Until recently, the B/CYDVs have also been classified into two Subgroups corresponding to broad differences in genomic structure, particularly in the region coding for the RNA-dependent RNA polymerase (ORF2; FIG. 1). Subgroup I viruses, which have polymerases that share features with carmoviruses, include BYDV-PAV, BYDV-MAV, and BYDV-SGV. Subgroup II viruses, which show greater similarity to sobemoviruses, include BYDV-RMV, CYDV-RPV, and BYDV-GPV (Koonin and Dolja, 1993; Miller et al., 1995; Wang et al., 1998). Although the Subgroup terminology is not used in the current B/CYDV classifications, these two groupings of B/CYDV reflect genomic differences assays can reliably distinguish. Thus, as used herein, “Subgroup I” refers to BYDV-PAV, BYDV-MAV, and BYDV-SGV viruses, and “Subgroup II” refers to BYDV-RMV, CYDV-RPV, and BYDV-GPV viruses.

In susceptible hosts, a B/CYDV infection typically impairs phloem function and causes stunting and reduction of seed production (Burnett, 1990; Esau, 1957). Infected plants may show characteristic purple or yellow foliar discoloration, or none at all (Irwin and Thresh, 1990). Since their identification in 1951 (Oswald and Houston, 1951), the B/CYDVs have been found to infect more than 150 different Poaceae species (D'Arcy, 1995) and to cause significant yield loss in cereals worldwide (Burnett, 1990; Lister and Ranieri, 1995). More recently, studies have found that the B/CYDVs are also common in natural grasslands (Guy et al., 1987; Power and Remold, 1996), and this finding has raised questions about the roles of these viruses in shaping natural grassland dynamics (Malmstrom, 1998).

Despite these intriguing findings, the study of B/CYDVs in natural systems has been limited by the lack of efficient diagnostic techniques for use with field assays of wild hosts. Determining the impact of virus pressure on natural host populations often requires monitoring and sampling marked individuals over a period of years. Environmental variability mandates that sampling must be extensive to be statistically useful, resulting in large sets of samples for analysis. At the same time, individual samples are typically small, both because true population sampling must include samples from seedlings and young plants, and because in studies requiring repeated sampling over time the sampling itself cannot become large-scale herbivory that damages or kills study plants. Diagnostics for use in natural systems must also be effective on samples from perennial grass hosts with fibrous leaves from which it can be harder to extract phloem sap and virus particles.

With cereals, enzyme-linked immunosorbent assays (ELISA) have traditionally been the preferred B/CYDV diagnostic for large sets of samples (Figueira et al., 1997; French, 1995; Lister and Rochow, 1979). However, ELISA typically requires relatively large (0.5-1 g) and “juicy” samples, which limits its usefulness for studies in which hosts are small or fibrous. Moreover, high quality B/CYDV antibodies for ELISA have become less available commercially over time, constraining the opportunities for conducting research with large numbers of samples or with specific viruses, such as BYDV-RMV and BYDV-SGV, for which antibody availability has been particularly limited.

Reverse-transcriptase polymerase chain reaction (RT-PCR) offers several advantages over ELISA (Henson and French, 1993; Martin et al., 2000). RT-PCR can detect B/CYDVs at low levels (Figueira et al., 1997; Canning et al., 1996), facilitating work with small samples and in low-titer hosts. In addition, RT-PCR eliminates concerns about reagent availability, and variability inherent across antibody batches, both critical issues in long-term studies of virus-host dynamics. The disadvantage of RT-PCR is the per-sample expense of the reagents, which can be a major concern for large-scale ecological studies. However, this disadvantage can be moderated if diagnostics are streamlined to maximize the amount of information gained in each procedure (Nassuth et al., 2000).

Robertson et al. (1991) developed a novel pair of short primers named Lu-1 and Lu-4 (SEQ ID NOS: 9 and 10) that generate a ˜533-bp fragment from a wide range of B/CYDVs, permitting one-step determination of broad B/CYDV status (infection +/−) in hosts. However, to gain more specific information about B/CYDV identity with this procedure requires the additional time and cost of a restriction digestion. In addition, because the primers are short, the protocol requires low temperatures with long annealing and extension times and high numbers of cycles. Under these conditions, the system is prone to producing nonspecific bands, as Robertson et al. note, particularly in negatives and, as we have found, in wild hosts.

The following are the details of references articles noted above:

### REFERENCES

Balaji, B., Bucholtz, D. B. and Anderson, J. M. (2003) Barley yellow dwarf virus and cereal yellow dwarf virus quantification by real-time polymerase chain reaction in resistant and susceptible plants. Phytopathology 93(11), 1386-1392.
Burnett, P. A., ed. (1990) World Perspectives on Barley Yellow Dwarf. CIMMYT, Mexico, D.F., Mexico.
Canning, E. S. G., Penrose, M. J., Barker, I. and Coates, D. (1996) Improved detection of barley yellow dwarf virus in single aphids using RT-PCR. J. Virol. Methods 56, 191-197.
Creamer, R. and Falk, B. W. (1989) Characterization of a nonspecifically transmitted CA RPV isolate of barley yellow dwarf virus. Phytopathology 79, 942-946.
D'Arcy, C. J. (1995) Symptomology and host range of Barley Yellow Dwarf. In: C. J. D'Arcy and P. A. Burnett (Eds), Barley Yellow Dwarf: 40 Years of Progress, Am. Phytopathol. Soc., St. Paul, Minn., pp. 9-28.
Dinesh-Kumar, S. P., Brault, V. and Miller, W. A. (1992) Precise mapping and in vitro translation of a trifinctional subgenomic RNA of barley yellow dwarf virus. Virology 187, 711-722.
Domier, L. L. (1995) Genome structure and function of barley yellow dwarf viruses. In: C. J. D'Arcy and P. A. Burnett (Eds), Barley Yellow Dwarf: 40 Years of Progress, Am. Phytopathol. Soc., St. Paul, pp. 181-201.
Drewes Milligan, K. L. and Cosper, E. M. (1994) Isolation of virus capable of lysing the brown tide microalga, Aureococcus anophagefferens. Science 266, 805-807.
Esau, K. (1957) Phloem degeneration in Gramineae affected by the barley yellow-dwarf virus. Am. J. of Bot. 44, 245-251.
Figueira, A. R., Domier, L. L. and D'Arcy, C. J. (1997) Comparison of techniques for detection of barley yellow dwarf virus—PAV-IL. Plant Dis. 81 (11), 1236-1240.
French, R. (1995) Barley yellow dwarf: Diagnostic procedures and reagents. In: P. A. Burnett (Ed), Barley Yellow Dwarf: 40 Years of Progress, Am. Phytopathol. Soc., St. Paul, pp. 293-305.
Gilbert, G. S. (2002) Evolutionary ecology of plant disease in natural ecosystems. Ann. Rev. Phytopathol. 40, 13-43.
Gray, S. and Gildow, F. E. (2003) Luteovirus-aphid interactions. Ann. Rev. Phytopathol. 41, 539-66.
Guy, P. L., Johnstone, G. R. and Morris, D. I. (1987) Barley yellow dwarf virus in, and aphids on, grasses (including cereals) in Tasmania. Aust. J. of Agric. Res. 38, 39-152.
Harper, J. L. (1977) The Population Biology of Plants. Academic, London.
Henegariu, O., Heerema, N. A., Dlouhy, S. R., Vance, G. H. and Vogt, P. H. (1997) Multiplex PCR: Critical parameters and step-by-step protocol. BioTechniques 23, 504 511.
Henson, J. and French, R. (1993) The polymerase chain reaction and plant disease diagnosis. Ann. Rev. Phytopathol. 143, 369-373.
Irwin, M. E. and Thresh, J. M. (1990) Epidemiology of barley yellow dwarf: a study in ecological complexity. Ann. Rev. Phytopathol. 28, 393-424.
Koev, G. and Miller, W. A. (2000) A positive-strand RNA virus with three very different subgenomic RNA promoters. J. Virol. 74(13), 5988-5996.
Koonin, E. V. and Dolja, V. V. (1993) Evolution and taxonomy of positive-strand RNA viruses: implications of comparative analysis of amino acid sequences. Crit. Rev. Biochem. Mol. Biol. 28(5), 375-430.
Lister, R. M. and Ranieri, R. (1995) Distribution and economic importance of barley yellow dwarf. In: P. A. Burnett (Ed), Barley Yellow Dwarf: 40 Years of Progress, Am. Phytopathol. Soc., St Paul, pp. 29-53.
Lister, R. M. and Rochow, W. F. (1979) Detection of barley yellow dwarf virus by enzyme-linked immunosorbent assay (ELISA). Phytopathology 69, 649-654.
Lister, R. M. and Rochow, W. F. (1988) Anomalies in serological and vector relationships of MAV-like isolates of barley yellow dwarf virus from Australia and the USA. Phytopathology 78, 766-770.
Malmstrom, C. M. (1998) Barley yellow dwarf virus in native California grasses. Grasslands 8(4), 1,6-10.
Martin, R. R., James, D. and Levesque, C. A. (2000) Impacts of molecular diagnostic technologies on plant disease management. Ann. Rev. Phytopathol. 38, 207-39.
Miller, W. A., Dinesh-Kumar, S. P. and Paul, C. P. (1995) Luteovirus gene expression. Crit. Rev. Plant Sci. 14(3), 179-211.
Nassuth, A., Pollari, E., Helmeczy, K., Stewart, S. and Kofalvi, S. A. (2000) Improved RNA extraction and one-tube RT-PCR assay for simultaneous detection of control plant RNA plus several viruses in plant extracts. J. Virol. Methods 90, 37-49.
Nienhaus, F. and Castello, J. D. (1989) Viruses of forest trees. Ann. Rev. Phytopathol. 27, 165-186.
Oswald, J. W. and Houston, B. R. (1951) A new virus disease of cereals, transmissible by aphids. Plant Dis. Report. 35(11), 471-475.
Plumb, R. T. (1974) Properties and isolates of barley yellow dwarf virus. Ann. Appl. Biol. 77, 87-91.
Power, A. G. and Remold, S. (1996) Incidence of barley yellow dwarf virus in wild grass populations: implications for biotechnology risk assessment. In: M. Levin, C. Grim and S. Angle (Eds), Biotechnology Risk Assessment., Vol. Proceedings of the Biotechnology Risk Assessment Symposium, Jun. 23-26, 1996, Balmar, Gaithersburg, Md., USA.
Robertson, N. L., French, R. and Gray, S. M. (1991) Use of group specific primers and the polymerase chain reaction for the detection and identification of luteoviruses. J. Gen. Virol. 72, 1473-1477.

Wang, M.-B., Cheng, Z., Keese, P., Graham, M. W., Larkin, P. J. and Waterhouse, P. M. (1998) Comparison of the coat protein, movement protein and RNA polymerase gene sequences of Australian, Chinese, and American isolates of barley yellow dwarf virus transmitted by Rhopalosiphum padi. Arch. Virol. 143, 1005-1013.
Wommack, K. E. and Colwell, R. R. (2000) Virioplankton: viruses in aquatic ecosystems. Microbiol. Mol. Biol. Rev. 64(1), 69-114.
Yount, D. J. and Carroll, T. W. (1983) Barley yellow dwarf luteoviruses in Montana cereals. Plant Dis. 67, 1217-1222.

### SUMMARY OF THE INVENTION

In one embodiment, the present invention is a method for detecting the presence or absence of a Subgroup I and a Subgroup II barley or cereal yellow dwarf virus in a biological sample by use of an amplification reaction, such as the polymerase chain reaction (PCR) or more specifically reverse transcriptase polymerase chain reaction (RPCR). Preferably, this method comprises the steps of isolating RNA from a biological sample and creating cDNA from the isolated RNA. The cDNA is exposed to oligonucleotide primers. A first primer pair, including a first forward primer and a first reverse primer, hybridizes with and selectively amplifies Subgroup I barley or cereal yellow dwarf viruses in the sample, and a second primer pair, including a second forward primer and the first reverse primer, hybridizes with and selectively amplifies Subgroup II barley or cereal yellow viruses. The sample is then examined to determine whether an amplification product exists for the oligonucleotide fragment produced by either primer pair. Preferably, the fragments are examined by electrophoresis on an agarose gel and visualized under UV illumination. The fragments produced by the first primer pair differ in size from the fragments produced by the second primer pair, and, therefore, the sample can be identified as containing either or both a Subgroup I and/or a Subgroup II barley or cereal yellow dwarf virus.

The present invention additionally includes a method for evaluating a biological sample not only to detect the presence or absence of a Subgroup I and a Subgroup II barley and cereal yellow dwarf virus, but also to identify the species of Subgroup I barley or cereal yellow dwarf virus suspected to be contained in the sample. Preferably, this method comprises the steps of isolating RNA from a biological sample and creating cDNA from the isolated RNA. The cDNA is exposed to oligonucleotide primer pairs that anneal with and selectively amplify Subgroup II barley and yellow dwarf viruses and Subgroup I barley yellow dwarf viruses BYDV-PAV, BYDV-MAV, and BYDV-SGV. The sample is exposed to the primer pairs under conditions that will allow for the production of amplification products if cDNA from a Subgroup II or any of the Subgroup I viruses is present in the sample. The sample is then examined to determine whether an amplification product exists for a Subgroup II virus or for any of the Subgroup I viruses.

The present invention also includes a set of primers for detecting the presence of Subgroup I and Subgroup II barley or cereal yellow dwarf viruses. This kit has a primer multiplex with a first primer pair of oligonucleotide primers, including a first forward primer and a first reverse primer, which anneal to and selectively amplify Subgroup I barley or cereal yellow dwarf viruses. This kit also has a second pair of oligonucelotide primers, including a second forward primer and the first reverse primer, which can anneal to and selectively amplify a Subgroup II barley or cereal yellow dwarf virus. The present invention also includes a kit having PCR primer pairs that not only can selectively hybridize with and detect Subgroup I and Subgroup II barley and yellow dwarf viruses, but also can selectively hybridize with Subgroup I barley yellow dwarf viruses BYDV-PAV, BYDV-MAV, and BYDV-SGV, and detect the presence of each species of Subgroup I virus.

More preferably, the RNA from a biological sample (after reverse transcription to cDNA) is exposed to a primer pair selected from the group of SEQ ID NOS: 1-8. A preferred oligonucleotide pair (forward and reverse) of primers to detect Subgroup I barley and cereal yellow viruses are nucleotides of SEQ ID NO: 2 (forward) and SEQ ID NO: 1 (reverse). Preferably, SEQ ID NO: 3 and/or 4 (forward) and SEQ ID NO: 1 (reverse) are used to detect Subgroup II barley and cereal yellow dwarf viruses. To distinguish among Subgroup I barley and cereal yellow viruses, the following primer pairs are preferred: to detect BYDV-PAV, SEQ ID NO: 5 (forward) and SEQ ID NO: 1 (reverse); to detect BYDV-SGV, SEQ ID NO: 2 (forward) and SEQ ID NOS: 6 and/or 7 (reverse); and to detect BYDV-MAV, SEQ ID NO: 8 (forward), and SEQ ID NO: 1 (reverse).

### BRIEF DESCRIPTION OF THE DRAWING

FIG. 1 is a schematic drawing of target sequences for primers and approximate size of fragments produced for Subgroup I B/CYDVs (top) and Subgroup II B/CYDVs (bottom).

FIG. 2 illustrates a UV illumination of an agarose gel electrophoresis demonstrating amplification of multiplex RT-PCR assays of Subgroup I and Subgroup II viruses produced using a first multiplex primer set (Panel A) and a second multiplex primer set (Panel B). The drawing shows bands for various isolates of the viral species.

FIG. 3 illustrates a UV illumination of an agarose gel electrophoresis demonstrating amplification of multiplex RT-PCR assays of Subgroup I and Subgroup II viruses produced using a first multiplex primer set (Panel A) and a second multiplex primer set (Panel B). The drawing shows bands for various isolates of the viral species.

FIG. 4 illustrates a UV illumination of an agarose gel electrophoresis demonstrating amplification of multiplex RT-PCR assays of Subgroup I and Subgroup II viruses produced using a first multiplex primer set (Panel A) and a second multiplex primer set (Panel B). The drawing shows bands for various isolates of the viral species.

### DETAILED DESCRIPTION OF THE INVENTION

The present invention may be understood more readily by reference to the following detailed description of specific embodiments and the Example, Tables, and Sequence Listing included hereinafter.

The Sequence Listing contained on “MULTIPLEXED RT-PCR ASSAY FOR DETECTION AND SEPARATION OF BARLEY AND CEREAL YELLOW DWARF VIRUSES,” with file title “MIC37 PP321 Sequence listing.txt” is incorporated-by-reference. This compact disc (attached hereto) was created on Jun. 9, 2005, and is 2.00 kilobytes.

As used in the application, “a” can mean one or more, depending on the context with which it is used. The acronym “PCR” is used interchangeably with “polymerase chain reaction.” The acronym “RT-PCR” is used interchangeably with “reverse transcriptase-polymerase chain reaction.” The term “oligonucleotide,” as used in the application, refers to primers, probes, and oligomer fragments to be detected. The term “kit,” as used herein, is intended to mean a set of items (e.g., a set of primers).

As used in the application, the term “primer” refers to an oligonucleotide whether natural or synthetic, which is capable of acting as a point of initiation or synthesis when placed under conditions in which synthesis of a primer extension product which is complementary to a nucleic acid strand is induced. For example, in the presence of nucleotides and an inducing agent such as DNA polymerase or reverse transcriptase, and at a suitable temperature and pH, the primer acts as a point of initiation for synthesis by reverse transcriptase of an RNA.

As used in the application, the terms “multiplex” and “multiplexed” refer to utilizing different primers that are capable of producing more than one specific nucleic acid sequence from a mixture of nucleic acids. For example, if two different specific nucleic acid sequences are to be produced, at least three different primers must be utilized, i.e., two forward and one reverse or two reverse and one forward.

In general, the present invention is an assay for barley and cereal yellow dwarf viruses that are detected through amplification of nucleotide sequences specific to these viruses. PCR is used to amplify a segment of the B/CYDV cDNA that is flanked by known stretches of DNA sequences at each end. Two primers bind to these known flanking sequences in a series of in vitro reactions catalyzed by an inducing agent such as DNA polymerase. The template DNA is first denatured by heating in the presence of a large molar excess of each of the two primers and four dNTPs. The mixture is then cooled to a temperature allowing the primers to anneal to their target sequences. Then, the annealed primers are extended by the DNA polymerase. The cycle of denaturation, annealing, and DNA synthesis is repeated (up to 50 times) to “amplify” and produce multiple copies of the target sequence. The large copy number of the target sequence allows for detection after the PCR reaction, usually by agarose or polyacrylamide gel electrophoresis and staining of the DNA. PCR is disclosed in U.S. Pat. Nos. 4,683,195 and 4,683,202.

The present invention includes two multiplexed RT-PCR assays that can detect and distinguish among different barley and cereal yellow dwarf viruses. A first multiplex can distinguish between Subgroup I and Subgroup II B/CYDVs by producing either an approximate 830 base pair fragment amplified from primers having SEQ ID NO: 1 and SEQ ID NO: 2 or, alternatively, by producing an approximate 372 base pair fragment amplified using primers of SEQ ID NOS: 1 (reverse) and 3 and/or 4 (forward). Better results may be obtained using both primers of SEQ ID NOS: 3 and 4. The 830 base pair fragment indicates the presence of any of the known Subgroup I barley and cereal yellow viruses BYDV-PAV, BYDV-MAV, and BYDV-SGV; while the 372 base pair fragment indicates the presence of any of the known Subgroup II barley or cereal yellow dwarf viruses CYDV-RPV, BYDV-RMV, and BYDV-GPV. A second multiplex can distinguish between Subgroup I and Subgroup II B/CYDVs as described with the first multiplex and also can produce two additional fragments which differentiate between the species of Subgroup I B/CYDVs. The second multiplex includes primers of SEQ ID NOS: 1-4 (which were used in the first multiplex) with the addition of primers of SEQ ID NOS: 5 (forward) and 6 and/or 7(reverse), producing a 590 base pair fragment for BYDV-PAV and a 254 base pair fragment for BYDV-SGV. Better results may be obtained using both primers of SEQ ID NOS: 6 and 7. To confirm mixed infections of Subgroup B/CYDVs, an additional fragment that identifies BYDV-MAV may be produced in a separate PCR reaction using primers of SEQ ID NOS: 1 and 8.

The two multiplex assays of the present invention fulfill the critical need for a streamlined diagnostic procedure for B/CYDVs that can be cost-effectively applied to a large number of small samples. These assays are useful not only in the basic diagnosis of B/CYDVs, but also for studies examining the ecological roles of B/CYDVs in natural systems for longer term epidemiological studies of grasses and cereals. These assays also simultaneously detect and distinguish different B/CYDVs, without the use of restriction digests. Further, the use of primer multiplexes in the present invention increases the amount of information to be gained in a single RT-PCR procedure.

In a preferred embodiment of the invention, RNA from sample B/CYDVs is reverse transcribed using reverse transcriptase and also a reverse primer (Yan-R: SEQ ID NO: 1) which reverse primer is also is used to reverse prime the fragments in the first and second multiplex assays.

The multiplexed assays were developed using plant tissue infected with known North American isolates. Samples included tissue that was fresh, frozen, or dried. Where possible, the tested isolates were matched with GenBank sequences as indicated in Tables 1 and 2.

Table 1 shows the complementarity of Yan-R reverse primer to available Subgroup I sequences (underlining). The symbol “˜” indicates a region where sequence data is not available (some GenBank sequences and isolates may be redundant).

TABLE 1

| | Gel | Accession | | |
|---|---|---|---|---|
| Isolate | Code | Num. | Origin | Sequence (5′ to 3′) |
| PAV(1) | P1, | See ¶ below | | CAAATAGGTAGACTCCTCAACA |
| | P4 | | | |
| PAV(2) | | See ¶ below | | CAAATAGGTAAACTCCTCAACA |
| PAV(3) | | See ¶ below | | CAAATAGGTAGAC˜˜˜˜˜˜˜˜˜ |
| PAV(4) | | See ¶ below | | CAAATAG˜˜˜˜˜˜˜˜˜˜˜˜˜˜˜ |
| PAV(5) | | See ¶ below | | AAAATAG˜˜˜˜˜˜˜˜˜˜˜˜˜˜˜ |
| PAV(6) | | See ¶ below | | GAAATAG˜˜˜˜˜˜˜˜˜˜˜˜˜˜˜ |
| MAV(1) | | See ¶ below | | CAAATAGGTAGACTCCTCAACA |
| MAV(2) | MI | See ¶ below | | CAAATAG˜˜˜˜˜˜˜˜˜˜˜˜˜˜˜ |
| SGV-TX | | U06866 | Texas | CAAATAGGTAGACCCCTCGCCG |
| SGV-NY | S1 | U06865, | New York | TAAATAGGTAGACCCCTCGCCG |
| | | AY541039 | | |
| primer | | Yan-R | | GTTTATCCATCTGAGGAGTTGT |
| | | (3′ to 5′) | | |
| | | Complement | | CAAATAGGTAGACTCCTCAACA |

Accession numbers for various isolates identified in Table 1 are as follows (with accession numbers for complete sequences shown in bold): PAV(1): AF218798 (New York), AF235167 (Illinois), AF391101(France), AJ007491, AJ007492, AY040344 (France), AY040343 (France), D11032/D01214 (Indiana), NC_{—}002160 (New York), NC_{—}004750 (Australia), U12928 (New York), X07653 (Australia); PAV (2): D85783 (Japan); PAV(3): AF213147 (Illinois), AF213148, AF213149, AF213150, AF213151, X56050 (New York); PAV(4): AF192967 (China), AJ007921 (Morocco), AJ007922 (Morocco), AJ007923 (Morocco), AJ007925 (Morocco), AJ007928 (Morocco), AJ007929 (Morocco), AJ223586 (France), AJ23587 (France), AJ223588 (France), AJ223589 (France), AJ295639 (Greece), AY167108 (France), AY167109 (France), L19471(Washington), L19504, M21347 (Australia), X17261 (Indiana), U29604; PAV(5): AJ007918 (Morocco), AJ007919 (Morocco), AJ007920 (Morocco), AJ007924 (Morocco), AJ007926 (Morocco), AJ007927 (Morocco), X17259 (New York); PAV (6): L10356 (China); MAV(1): AF338909 (China), AY220739 (China), D11028/D01213 (New York), NC_{—}003680, NC_{—}004666 (China); MAV(2): X17260 (New York), X53174.

Table 2 shows the complementarity of Yan-R reverse primer to available Subgroup II sequences (underlining). The “˜” symbol indicates a region where sequence data is not available (some GenBank sequences and isolates may be redundant). Bold accession numbers indicate complete sequences and the symbol “*” indicates the isolate probably is the same.

TABLE 2

| | Gel | Accession | | |
|---|---|---|---|---|
| Isolate | Code | Num. | Origin | Sequence (5′ to 3′) |
| | | D10206, | | |
| | | D01013 | | |
| RPV-NY | R6 | L25299, | New York | AAAATAGGTAGACGCGGAACCC |
| | | NC-004751* | | |
| RPV | | AF235168 | Mexico | GAAATAGGTAGACGCGGAACCC |
| RPV | | NC-002198 | Mexico | GAAATAGGTAGACGCGGAACCC |
| RPV | | AF020090 | Australia | AAAATAG˜˜˜˜˜˜˜˜˜˜˜˜˜˜˜ |
| RPV-NY | R6 | X17259* | New York | AAAATAG˜˜˜˜˜˜˜˜˜˜˜˜˜˜˜ |
| GPV | | AF216863 | | GAAATAG˜˜˜˜˜˜˜˜˜˜˜˜˜˜˜ |
| GPV-CN | | L10356 | China | GAAATAG˜˜˜˜˜˜˜˜˜˜˜˜˜˜˜ |
| RMV-MT | | Unpub. from | Montana | GAAATAGGTAGACGGAGCTTCC |
| | | R. French | | |
| RMV-NY | V1, | Unpub. from | New York | GAAATAGGTAGACGGAGCATCT |
| | V2 | R. French | | |
| RMV-like | | Z14123 | Illinois | CAAATAGGTAGAC˜˜˜˜˜˜˜˜˜ |
| RMV- | | L12757 | Montana | CAAATAG˜˜˜˜˜˜˜˜˜˜˜˜˜˜˜ |
| Montana | | | | |
| RMV- | | L12758 | Montana | CAAATAG˜˜˜˜˜˜˜˜˜˜˜˜˜˜˜ |
| Montana | | | | |
| primer | | Yan-R | | GTTTATCCATCTGAGGAGTTGT |
| | | (3′ to 5′) | | |
| | | Complement | | CAAATAGGTAGACTCCTGAACA |

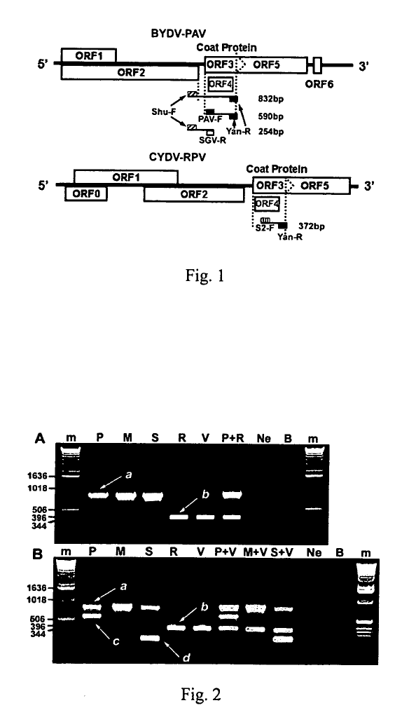

To design and evaluate the multiplex primers, aligned B/CYDV sequences from the NCBI database were compared using Genetic Computer Group program, Version 10.1 (University of Wisconsin, Madison, Wis.). Presently, GenBank contains complete sequences for seven BYDV-PAVs, four BYDV-MAVs, and two to four CYDVs (http://www.ncbi.nlm.gov). These counts are approximate because there is some redundancy among the isolates sequenced and sequences submitted. For the remaining three viruses (BYDV-SGV, BYDV-GPV, and BYDV-RMV), GenBank contains a limited number of partial sequences, and these focus on ORF3 (FIG. 1), the coat-protein coding region, which is among the most highly conserved regions of the B/CYDV genome. Thus, the initial evaluation focused in the ORF3 and adjoining ORF2 and ORF5 regions. (FIG. 1). FIG. 1 shows a genomic map of Subgroup I (top) and Subgroup II (bottom) B/CYDVs. Candidate primers were further analyzed with the Primer3 (http://www.broad.mit.edu/cgi-bin/primer/primer3_www.cgi) and Oligo 6 (Molecular Biology Insights, Inc.) software packages, and then tested extensively in different combinations in the laboratory.

The multiplexes of the present invention are built around a universal reverse primer that is used both for reverse transcription and amplification. One initial candidate for this primer was Lu-4-R, the 14-bp reverse primer designed by Robertson et al. (1991) that corresponds to a highly conserved region at the 3′ end of ORF3 (SEQ ID NO: 10). However, Lu-4-R is too short to be compatible with the forward primers (24-25 bp) required for discriminating between Subgroups I and II, and between the three members of Subgroup I. Using longer primers and higher annealing temperatures is also desirable because doing so helps eliminate the non-specific banding common with the Lu system.

Ultimately, a 22 bp Yan-R primer (SEQ ID NO: 1) was selected as a universal primer.

Yan-R is a modification of Lu-4-R that extends 9 bp further in the 5′ direction into ORF 5 and is one nucleotide shorter in the 3′ direction (FIG. 1). Table 3 illustrates the universal Yan-R primer and other primers examined (bold indicates overlap with Lu-4-R).

TABLE 3

| | | | | |
|---|---|---|---|---|
| Primer | Primer Sequence (5′ to 3′) | 5′ Position | Specificity | Product Size |

| | | | | |
|---|---|---|---|---|
| Yan-R | TGTTGAGGAGTCTACCTATTTG | PAV AF235167: 3475 | General | |
| | | MAV D11028: 3436 | | |
| | | SGV U06866: 606 | | |
| | | RPV D10206: 4310 | | |
| | | RMV L12757 | | |
| Shu-F | TACGGTAAGTGCCCAACTCC | PAV AF235167: 2645 | Subgroup I | With Yan-R: |
| | | MAV D11028: 2609 | | ˜830 bp, occ. faint |
| | | SGV AY540130: 532 | | ˜300 bp in BYDV-PAV |
| | | | | samples |
| S2a-F | TCACCTTCGGGCCGTCTCTATCAG | RPV D10206: 3937 | Subgroup II | With Yan-R: ˜372 bp |
| S2b-F | TCACCTTCGGGGCGTCTCTTTCTG | RMV L12757: 151 | | With Yan-R: ˜372 bp |
| PAV-F | ACCTAGACGCGCAAATCAAA | PAV AF235167:2881 | PAVs | With Yan-R: ˜590 bp |
| SGV-R | ACATTTCTTCGTGTGTTGCG | SGV AY540130: 784 | SGVs | With Shu-F: ˜254 bp |
| | ACATTTTTGCGTGCGTTGCG | SGV U06865: 41 | | With Shu-F: ˜254 bp |
| MAV2-F | AATAACCGCAGGAGAAATGG | MAV D11028: 2843 | MAVs | With Yan-R: ˜590 bp |

The first multiplex system of the present invention is designed to produce two distinct fragments that discriminate between Subgroup I and II viruses. Because of the strong ORF2 differences between these two Subgroups, a 20-bp forward Subgroup I primer named “Shu-F” (SEQ ID NO: 2) was designed to match a target sequence at the 3′ end of ORF2 unique to the Subgroup I viruses, BYDV-PAV, BYDV-MAV and BYDV-SGV (FIG. 1; Table 3). Because no BYDV-SGV sequences were initially available in the ORF2 region, part of this genomic region was sequenced from four isolates (S1-S4 in FIG. 4) before completing the primer design. The sequences were manually edited and then assembled using the Sequencher™ (GeneCodes Corp. Ann Arbor, Mich.). The sequences have been deposited in the NCBI nucleotide database (Accession numbers AY540130, AY541037, AY541038, AY541039 http://www.ncbi.nlm.gov).

To detect the Subgroup II B/CYDVs and produce a fragment distinct from that produced by Shu-F, two closely related 24-bp forward Subgroup II primers named “S2a-F” and S2b-F” (SEQ ID NOS: 3 and 4) were designed. The S2a-F and S2b-F primers have high homology to an ORF3 target sequence unique to the Subgroup II viruses (FIG. 1; Table 3).

The second multiplex system includes the primers from the first multiplex (Yan-R and Shu-F) and was designed to further discriminate between the three members of Subgroup I (BYDV-PAV, BYDV-MAV, BYDV-SGV). Because of strong sequence similarity within the region immediately upstream of Yan-R, one genomic location was identified (at the 5′-end of ORF3) for which such primers were designed (FIG. 1). After extensive screening of compatibility of primer candidates, a 20 bp “PAV-F” forward primer (SEQ ID NO: 5) and 20 bp “SGV-R” reverse primers (SEQ ID NOS: 6 and 7) were developed for this location (FIG. 1; Table 3). Two forward primers at this site would have produced fragments indistinguishable from each other, so by using one forward and one reverse, the site's diagnostic usefulness was doubled. Because adding a completely new primer pair (new forward plus reverse) introduced unwanted primer interactions, and a third primer at this site that produced a fragment of a length different than the others was not determined, a BYDV-MAV-specific primer is not included in the second primer multiplex. Instead, the second multiplex is designed so that BYDV-MAV samples are identified by the presence of a single Subgroup I Shu fragment (i.e., the absence of any fragment produced by either the PAV-F or SGV-R primers). Alternatively, a 20 bp “MAV-F” forward primer (SEQ ID NO: 8) has been designed for use with Yan-R in a subsequent assay.

The first multiplex assay is generally somewhat easier to optimize and run cleanly than the second multiplex assay, because of the difference in the number of primer pairs, but the second assay offers the reward of additional information. If questions about the diagnosis of a particular sample arise, any of the primer pairs may be rerun singly on the cDNA from the original reverse transcription product.

Because the first and second multiplexes are based on a single reverse transcription (RT) step with a universal Yan-R reverse primer, variations of the PCR protocol can be easily adjusted, without requiring the time and expense of re-running the RT step. Using a universal reverse primer and a single RT step also lowers the chances that sample RNA will be contaminated, or degraded by repeated freeze/thawing, because each RNA sample will typically be handled only once. In addition, interactions between multiple reverse primers in the RT are avoided, and interactions caused by carryover of RT primer to PCR are minimized.

The Yan-R performs well as a universal reverse primer (as set forth in the Example below). At its 3′ end, the Yan-R overlaps the Lu-4-R and this portion is highly complementary to conserved regions in both the Subgroup I and II viruses (Tables 1 and 2). The 5′ end of Yan-R shows close to 100% complementarity with all GenBank BYDV-PAV and BYDV-MAV sequences (Table 1). With BYDV-SGV and the Subgroup II viruses, there is some notable mismatch at the 5′ end, but good amplification still results (FIGS. 4 and 5; Tables 1 and 2). Removing the 3′ G, where some mismatch occurs, or adding the additional 3′ G found in Lu-4-R, has a deleterious impact on primer performance, typically resulting in the formation of unwanted extra bands (data not shown). Because Yan-R is 8 bp longer than Lu-4-R, it can be successfully paired with the other longer primers needed for these multiplexes and run with higher annealing temperatures and shorter extension periods, reducing PCR time and producing cleaner gels.

The additional multiplex primers also perform well (as set forth in the Example) with the Yan-R primer, the Shu-F primer and S2-F (a and b) primers consistently and cleanly detected Subgroup I and II viruses, respectively. In combination with these primers in the second multiplex, the PAV-F and the SGV-R primers reliably differentiated between BYDV-PAV, BYDV-MAV, and BYDV-SGV. Because the Shu-F primer is homologous to an ORF2 sequence, its effectiveness may theoretically be reduced in tissues containing high levels of sgRNA1, which only encompasses ORFs 3-5 (Dinesh-Kumar et al., 1992; Koev and Miller, 2000). It is possible that by competing with the gRNA as a site for Yan-R, the sgRNA1 might reduce transcription of the genomic fragment containing the Shu-F site. However, no evidence of this phenomenon was seen.

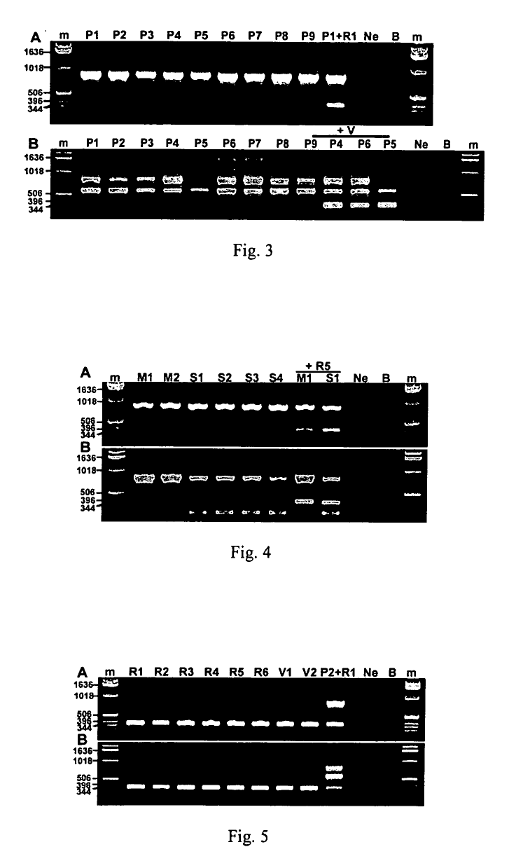

The multiplexes of the present invention can be used successfully on fresh tissue or on tissue that has been kept dry or frozen for some time. The named isolates labeled P6, P7, S4, R1, R2, and R3 (see FIGS. 3-5), for example, were extracted from dry tissue that had been stored at −20° C. for 16-21 yr. Also, fragments from BYDV-PAV from tissue dried at 65° C. and stored at room temperature for 10 years have been amplified (data not shown).

Based on the sequence information currently available, it is expected that the assays will have a relatively large range of geographic applicability. All BYDV-PAVs sequenced to-date, representing a broad range of geographic origins, appear to be compatible with the Yan-R, although for some isolates only partial sequences are available (Table 1). The seven GenBank BYDV-PAV sequences containing ORF2 show 100% homology with Shu-F, indicating applicability across three continents. [AF218798 PAV-129 (New York); AF235167 PAV-Ill (Illinois); D11032/D01214 P-PAV (Indiana); D85783 (Japan); NC_{—}002160 (New York); NC_{—}004750 (Australia); X07653 (Australia)]. Likewise, the same broad set of BYDV-PAV genomes shows high homology between PAV-F and its intended ORF3 target sequence (the homology is 100%, except for AF218798, D85783, and NC_{—}002160, which show single-bp mismatches in the center).

Fewer sequences are available for BYDV-MAV, but the GenBank set from New York and China suggests that Yan-R (Table 1), Shu-F, and MAV2-F should be broadly effective with this virus. D01213/D11028 (New York) and NC_{—}003680 (New York) are 100% homologous to Shu-F; NC_{—}004666 (China) and AY220739 (China) show a single-bp mismatch towards the 5′-end; AF338909 (China), AY220739 (China), and NC_{—}004666 (China) are 100% homologous to MAV2-F; and D11028/D01213 (New York), NC_{—}003680, and X53174 are homologous to MAV2-F except for single mismatches at positions 4 and 15, from the 5′ end.

For BYDV-SGV, the genomic information is strongly limited and no complete sequences are currently available. Experimental results (FIG. 4) and the partial sequences available indicate that Yan-R (Table 1), Shu-F, and SGV-R are compatible with known isolates, but more sequencing information is needed to determine the degree of geographic variability within this virus. S1 SGV AY541039 (New York), S2 SGV-I T4 AY540130, S3 SGV T2 AY541037, and S4 SGV-NY AY541038 (New York) are homologous to Shu-F except for single mismatches at positions 1 and 7, from the 5′ end; SGV1-R is 100% complementary to U06866 (Texas), S2 SGV-I T4 AY540130, and S3 SGV T2 AY541037. SGV2-R is 100% complementary to U06865 (New York), S1 AY541039 (New York), and S4 AY541038 (New York).

Among the Subgroup II viruses, complete GenBank sequences are currently available only for CYDV-RPV. The multiplex assays successfully amplified all the California and New York CYDV-RPV isolates tested (FIG. 5), one of which (FIG. 5, R6) has been sequenced (Table 2). This sequenced isolate shows the same pattern of complementarity with Yan-R as other GenBank sequences from Mexico and Australia (Table 2), so Yan-R should function well with these isolates too. The Subgroup II forward primer, S2a-F, is highly homologous to its intended CYDV-RPV target sequence across isolates in New York, Australia, and Mexico, and likely to be successful with other similar isolates. S2a-F shows 100% homology with the New York isolate (D10206, D01013, NC_{—}004751, L25299, also probably the same as X17259) and a single base-pair mismatch with the Australian (AF020090) and Mexican (AF235168, NC_{—}002198) isolate sequences.

The sequences for BYDV-RMV are much more limited. A New York BYDV-RMV is reliably detected with both multiplex assays (FIG. 5, V1, V2), so it is likely that other similar BYDV-RMVs will be detected as well (Table 2). The S2a-F primer, which is highly homologous to the target sequences in CYDV-RPV, shows a single bp mismatch with L12758 Montana (may be New York) and the unpublished RMV-NY sequence from Roy French and two mismatches with Z14123 Illinois. The S2b-F primer is 100% homologous to the target sequences in L12757 Montana and the corresponding unpublished sequence from Roy French. However, because BYDV-RMVs have a reputation for being variable and harder to diagnose (Yount and Carroll, 1983), more sequencing of a global range of samples is needed. Similarly, although BYDV-GPVs seem generally comparable to CYDV-RPVs at the primer sites (Table 2) (the BYDV-GPV sequences AF216863 and L10356 China show a single base-pair mismatch with S2a-F), more sequencing of these isolates would help determine the general applicability of the multiplex assays as currently configured. Sequence data for ORF2 and ORF5 would be particularly helpful, because these would facilitate the development of additional multiplex options.

The rate of false negatives with the multiplexes of the present invention will be a function of sample quality, laboratory technique, and the degree to which the genome of sampled viruses diverges from those known in GenBank. In general, RT-PCR is much more sensitive than ELISA (Figueira et al., 1997), and thus less prone to false negatives when virus concentrations are low. For example, Canning et al. found (1996) that RT-PCR was able to detect BYDV at concentrations 10-5 times lower than ELISA. However, degradation of sample tissue by fungi or by exposure to extreme heat or moisture will reduce the chances that sufficient viral RNA will remain for RT-PCR. In tests with named isolates, there were no false negatives when the protocol (as set forth in the Example below) was followed. Alternatively, false negatives may arise from errors in RT-PCR technique, most commonly from loss of the pellet during extraction, accidental introduction of RNase into reaction mixtures, or use of poor quality reagents.

In general, use of these multiplexes should reduce false negatives and increase the frequency with which B/CYDVs are detected. For example, the USDA ARS laboratory at Cornell University was unable to detect B/CYDVs in symptomatic bahiagrass (Paspalum notatum Flügge) from Florida, using serological methods (S. M. Gray, personal communication). In contrast, both the first and second multiplexes were able to successfully detect the presence of Subgroup II B/CYDVs, confirmed by sequencing as a virus most closely related to BYDV-RMV (data not shown).

For optimizing the multiplex assays of the present invention, users should verify that the primer balance is optimized for the tissue with which they are working, and that all fragments are produced equally well. Some adjustments in relative primer concentrations may be appropriate, if the relative virus titers in mixed infections are notably different than those described in the Example. In optimizing for the appearance of the 590-bp fragment in PAVs in the second multiplex, the upper 830-bp Shu fragment may become somewhat faint with some isolates (e.g., P5, FIG. 3, Panel B), but this is acceptable because the 590-bp fragment alone is necessary for diagnosis. For optimal use of the second multiplex to assess mixed infections of Subgroup I viruses, users should note that the presence of BYDV-MAV will be obscured in mixed infections including BYDV-PAV or BYDV-SGV because the second multiplex does not contain a BYDV-MAV-specific primer. If BYDV-MAV is common in the region from which the samples came, or if simple BYDV-MAV infections are detected in a sample set, users may wish to test samples positive for BYDV-PAV or BYDV-SGV for mixed infection with BYDV-MAV by rerunning the PCR from the original RT, using Yan-R and MAV2-F alone. Because the RT does not need to be repeated, the cost savings remain significant.

Further, because multiplex assays generally are more strongly affected by decline in dNTP activity than single PCR assays, dNTP aliquots for multiplex work should not be subjected to freezing and thawing more than three to five times (Henegariu et al., 1997).

In a preferred embodiment of the invention, no more than 1-2 μl of extracted RNA solution is used (more preferably about 1 μg of RNA), because RT-PCR sometimes is inhibited by foliar compounds carried over from RNA extraction, particularly in wild grasses or plants that are highly symptomatic. If inhibition appears to be a problem, the amount of extract used can be reduced. Alternatively, a different RNA extraction method that produces cleaner RNA can be used, such as the RNeasy Plant Mini Kit (QIAGEN, Valencia, Calif., USA). However, because the per-sample cost of kits like these is typically much greater than that of the TriReagent extraction method outlined here, mini kits are usually not practical for large numbers of samples or limited budgets.

If sample quality appears questionable (for example, if samples have been delayed in transit or have been collected from older tissue), users may test a preliminary set of samples for the presence of MDH or RubiscoL mRNAs, to assess the degree to which RNA values have been compromised (Nassuth et al., 2000). Even when mRNAs have been degraded, however, it may still be possible to detect genomic RNA that has been protected in virions.

These multiplexed RT-PCR assays offer several advantages over other diagnostic approaches and fulfill an identified need for enhanced molecular techniques in studies of virus ecology (Canning et al., 1996; Gilbert, 2002). Because the assays permit identification of B/CYDVs quickly and cost-effectively, they can be used to assess disease patterns in a wide suite of agricultural and natural populations. The multiplexes will be particularly useful for cases in which a large number of small samples must be analyzed; where high accuracy is demanded but costs must be contained; or where long-term analyses require the use of invariant reagents over a lengthy period, a requirement that is much easier to meet with known primers than with antibodies experiencing batch-to-batch variation. Along with new real-time RT-PCR approaches that allow in-depth analysis of selected individuals (Balaji et al., 2003), these multiplex assays will offer valuable new insights into the ecological dynamics of viruses across spatially diverse landscapes. B/CYDVs are well known pathogens of grasslands, but the contribution of viruses such as these to ecological dynamics in natural systems is only beginning to be explored.

It will be understood by those who practice the invention and those in ordinary skill in the art that various modifications and improvements may be made to the invention without departing from the spirit of the disclosed concept. The scope of protection afforded is to be determined by the claims in the breadth of interpretation allowed by the law. For example, it is contemplated that modification (e.g. single nucleotide substitutions, additions, or deletions) to the synthetic nucleic acids set forth in the sequence listing can be made which will not prevent these synthetic nucleic acids from annealing to the conserved target sequences from which they were derived. Such modified nucleic acids are still within the invention if they selectively hybridize with the sequence necessary for hybridization, i.e., the sequence complimentary to the primary sequence set forth.

### EXAMPLE

### Detention of Barley and Cereal Yellow Dwarf Viruses by RT-PCR Using Mixed Primers in Multiple Assays

The present invention is more particularly described in the following Example, which is intended as illustrative only, since numerous modifications and variations therein will be apparent to those skilled in the art.

Reverse Transcription (RT)

RNA concentration was determined by spectrophotometric analysis (DU Series 500 UV/Vis spectrophotometer, Beckman Coulter, Inc., Fullerton, Calif., USA). 1 μg of total RNA extract was used for reverse transcription with 0.2 μM of Yan-R primer and 1 μl of SuperScript II RNase H- Reverse Transcriptase (Invitrogen Life Technologies, Frederick, Md., USA), according to the manufacturer's instructions.

Polymerase Chain Reaction

A range of reagent concentrations and PCR conditions were tested for both multiplexes, to optimize their performance. The following represent the optimized protocols:

In both multiplex reactions, the total reaction volume was 20 μl. Each PCR reaction included 2 μl of RT product (˜100 ng), 1.5 mM MgCl2, 200 μM of each dNTP, 1× PCR Buffer and 1 Unit of AmpliTaq Gold DNA polymerase (PE Applied Biosystems, Foster City, Calif., USA). In the basic multiplex, the following primers were used: 0.2 μM Shu-F, 0.3 μM Yan-R, and 0.07 μM (total) of mixed S2a-F and S2b-F. In the enhanced multiplex, three additional primers were added to those used in the basic multiplex: 0.1 μM PAV-F, 0.15 μM SGV1-R, and 0.15 μM SGV2-R.

Samples were amplified using a Peltier Thermal Cycler (PTC-200, MJ Research, Watertown, Mass., USA). In both protocols, the AmpliTaq Gold DNA polymerase was first activated at 95° C. for 10 min. and then 35 PCR cycles were run. For the basic multiplex, the PCR conditions were: denaturation (95° C., 30 sec), annealing (60° C., 30 sec) and extension (72° C., 30 sec to 1 min), with a final extension (72° C., 7 min). For the enhanced multiplex, the PCR conditions were: denaturation (95° C., 30 sec), annealing (55° C., 45 sec) and extension (72° C., 1 min), followed with a final extension for 7 min at 72° C. The lower annealing temperatures and longer annealing and extension times used in the enhanced multiplex system are required because of the number of primers and competing reactions (Henegariu et al., 1997). To run the primer pairs singly, we used 0.2 μM of forward primer and 0.2 μM of reverse, with annealing at 55-60° C. for 30 sec and extension at 72° C. for 30 sec.

Analysis of Amplified Product

PCR products were analyzed by electrophoresis on a 1.25% agarose gel and visualized under UV illumination with a Bio-Rad gel documentation system (Bio-Rad Laboratories, Inc., Hercules, Calif., USA). Fragment sizes were determined by comparison with 1 kb DNA marker (Invitrogen Life Technologies™, Frederick, Md., USA).

Sequencing of PCR Products

Amplified DNA fragments were purified using QIAquick PCR Purification kit (QIAGEN, Valencia, Calif., USA), according to the manufacturer's instructions. The purified DNA fragments were submitted along with forward primer or reverse primer to the Genomics Technology Support Facility (Michigan State University, East Lansing, Mich., USA) for direct sequencing. Sequence identities were verified by a BLAST search of the NCBI nucleotide database (http://www.ncbi.nlm.nih.gov/blast).

Results

As shown in FIG. 2 (Panel A), the first multiplex reliably indicates the presence of Subgroup I and Subgroup II viruses. Specifically, the Shu-F forward primer (SEQ ID NO: 2) and Yan-R reverse primer (SEQ ID NO: 1) consistently produced a ˜830-bp fragment (identified as in FIG. 2 as “a”) from tissue infected with any of the Subgroup I viruses, BYDV-PAV, BYDV-MAV, or BYDV-SGV; and the S2-F forward primers (SEQ ID NOS: 3 and 4) and Yan-R reverse primer (SEQ ID NO: 1) consistently produced ˜372 bp fragments (identified in FIG. 2 as “b”) from tissue infected with CYDV-RPV or BYDV-RMV. When tested on mixed samples with both Subgroup I and II viruses, the first multiplex produced two fragments as expected (FIG. 2, Panel A).

As shown in FIG. 2 (Panel B), the second multiplex reliably indicates which of the three Subgroup I viruses is in the sample. Specifically, all Subgroup I samples showed the 830-bp Shu-F fragment, as in the first multiplex. BYDV-PAV samples produced a second distinctive ˜590-bp fragment (identified in FIG. 2 as “c”; FIG. 2, Panel B, lane 2) and the BYDV-SGV produced a second ˜254-bp fragment (identified in FIG. 2 as “d”; FIG. 2, Panel B, lane 4). As expected, the BYDV-MAV samples could be identified by the absence of a second fragment (FIG. 2, Panel B, lane 3).

The first and second multiplex assays were both robust in tests against a range of North American Subgroup I virus isolates (FIG. 3, Panels A and B). All nine BYDV-PAVs tested were reliably detected by both assays (FIG. 3, Panel A). In some BYDV-PAV isolates, a faint lower band at ˜300-bp is occasionally seen with Shu-F (e.g., P5, FIG. 3a), the result of some mispriming with Yan-R at a secondary site. This fragment is distinctly smaller than, and easily distinguished from, the 372-bp Subgroup II fragment produced by the S2-F. It can generally also be distinguished from the smaller ˜254-bp SGV fragment in the second multiplex. In addition, as shown in FIG. 4 (Panels A and B), the first and second multiplexes both correctly detected the two BYDV-MAVs tested (lanes 2 and 3), and all four BYDV-SGVs (lanes 4-7). The separation of the BYDV-PAVs, -SGVs, and -MAVs was clean, and no misidentification occurred.

Both multiplexes also reliably detected all the North American BYDV-RMV and CYDV-RPV Subgroup II samples tested, either singly (FIG. 5, Panels A and B, lanes 2-9) or in mixtures with Subgroup I viruses (FIG. 3, Panel A, lane 11; FIG. 3, Panel B, lanes 11-13; FIG. 4, Panels A and B, lanes 8 and 9; FIG. 5, Panels A and B, lane 10).

When Subgroup I and II viruses were jointly present in a sample, both viruses were detected by either assay (FIGS. 2-5). The second multiplex can also identify mixed infections containing both BYDV-PAV and BYDV-SGV, as indicated by the presence of three fragments: the 830-bp Shu fragment, the 590-bp PAV fragment, and the 254-bp SGV fragment. Because the second multiplex does not include a BYDV-MAV-specific primer, a mix of BYDV-PAV and BYDV-MAV will appear to be purely BYDV-PAV (830-bp Shu fragment, 590-bp PAV fragment) and a mix of BYDV-SGV and BYDV-MAV will appear to be purely BYDV-SGV (830-bp Shu fragment, 254-bp SGV fragment). Thus, to confirm the presence of BYDV-MAV in mixed infections, the BYDV-MAV-specific primer, MAV2-F (SEQ ID NO: 8), can be used with Yan-R in a second PCR from the same RT product. As shown in FIG. 4, the MAV2-F primer detected both BYDV-MAV isolates (M1, M2) and did not produce fragments from any BYDV-PAVs (P1-P9) or BYDV-SGVs (S1-S4). However, the MAV2-F primer is not a preferred replacement for PAV-F in the second multiplex, because the MAV2-F primer does not compete well with Shu-F.

Referring to FIGS. 2-5, the following are the gel codes for the isolates tested:

FIG. 2 shows gel analysis of B/CYDVs as follows: P: BYDV-PAV; M: BYDV-MAV; S: BYDV-SGV; R: CYDV-RPV; V: BYDV-RMV.

FIG. 3 shows gel analysis of North American BYDV-PAV isolates as follows: P1: PAV (Gray; U12928); P2: PAV-6 (Gray); P3: PAV-PH2a (Malmstrom; California); P4:

PAV-129 (Gray; AF218798); P5: PAV-PH2b (Malmstrom; California); P6: CA-PAV-2 T45 (Falk; California); P7: NY-PAV T52 (Falk; New York); P8: CA-PAV Jan. 24, 2000 (Falk; California); P9: PAV-129+PAV-6 (Gray). FIG. 3 also shows BYDV-RMV isolate (Gray; New York).

FIG. 4 shows gel analysis on North American BYDV-MAV and BYDV-SGV isolates, as follows: M1: MAV (Gray; X53174 New York); M2: MAV-CA TR6 14 1987 RV (Falk; California); S1: SGV (Gray; U06865, AY5413039 New York); S2: SGV-I T4 (Falk; AY540130); S3: SGV T2 (Falk; AY541037); S4: SGV-NY (Falk; AY541038; New York).

FIG. 5 shows gel analysis on North American CYDV-RPV and BYDV-RMV isolates, as follows: R1: RPV-CA T35 (Falk; California); R2: CA-RPV-2 T45 (Falk; California); R3: CA-RPV-4 (Falk; California); R4: RPV-NY (Falk; New York); R5: CA-RPV Aug. 20, 2003 (Falk; California); R6: RPV (Gray; D10206, D01013, L25299, NC-004751; probably also X17259); V1,V2: RMV (Gray; New York; unpublished sequence from R. French).

What is claimed is:

1. A method of detecting the presence of a nucleic acid of a Subgroup I or a Subgroup II barley or cereal yellow dwarf virus in a sample comprising the steps of:
(a) providing a sample suspected of containing one or more nucleic acids encoding a protein of a Subgroup I or a Subgroup II barley or cereal yellow dwarf virus;
(b) isolating nucleic acid from the sample provided in step (a);
(c) exposing cDNA created from the nucleic acid of step (b) to PCR reagents, which PCR reagents include a primer multiplex of a first oligonucleotide primer pair and a second oligonucleotide primer pair wherein the first primer pair can anneal to and selectively amplify a Subgroup I barley or cereal yellow dwarf virus nucleic acid sequence, and the second primer pair can anneal to and selectively amplify a Subgroup II barley or cereal yellow dwarf virus nucleic acid sequence; and
(d) detecting the presence of a Subgroup I barley or cereal yellow dwarf virus and a Subgroup II barley or cereal yellow dwarf virus after amplification under suitable conditions.

2. The method of claim 1 wherein the first primer pair includes a first forward primer and a first reverse primer and the second primer pair includes a second forward primer and the first reverse primer.

3. A method of detecting the presence or absence of a nucleic acid sequence of a Subgroup I barley or cereal yellow dwarf virus in a sample, comprising the steps of:
(a) providing a sample suspected of containing one or more nucleic acids encoding a protein of a Subgroup I barley or cereal yellow dwarf virus;
(b) isolating nucleic acid from the sample provided in step (a);
(c) exposing cDNA created from the nucleic acid of step (b) to PCR reagents, which PCR reagents include a primer multiplex of a first oligonucleotide primer pair and a second oligonucleotide primer pair wherein the first primer pair can anneal to and selectively amplify a BYDV-SGV Subgroup I barley yellow dwarf virus nucleic acid sequence, and the second primer pair can anneal to and selectively amplify a BYDV-PAV barley yellow dwarf virus nucleic acid sequence; and
(d) detecting the presence or absence of BYDV-PAV, BYDV-MAV, and BYDV-SGV barley yellow dwarf viruses after amplification under suitable conditions.

4. A kit for detecting the presence of a nucleic acid of a Subgroup I or Subgroup II barley or cereal yellow dwarf virus that is suspected of being contained in a sample, the kit comprising:
a first pair of oligonucleotide PCR primers which can anneal to and selectively amplify a Subgroup I barley or cereal yellow dwarf virus, and a second pair of oligonucleotide PCR primers which can anneal to and selectively amplify a Subgroup II barley or cereal yellow dwarf virus.

5. The kit of claim 4 wherein the first pair of primers includes a first forward primer and a first reverse primer and the second pair of primers includes a second forward primer and the first reverse primer.

6. A kit for detecting the presence or absence of a nucleic acid sequence of a Subgroup I barley or cereal yellow dwarf virus that is suspected of being contained in a sample, the kit comprising:
a first forward oligonucleotide primer, a second forward oligonucleotide primer, first reverse oligonucleotide primer, and a second reverse oligonucleotide primer, wherein the first forward primer and the second reverse primer can anneal to and selectively amplify a BYDV-SGV nucleic acid sequence, the second forward primer and the first reverse primer can anneal to and selectively amplify a BYDV-PAV nucleic acid sequence, and the first forward primer and the first reverse primer can anneal to and selectively amplify any Subgroup I barley or yellow dwarf virus nucleic acid sequence.

7. Two pairs of oligonucleotide primers for a polymerase chain reaction to amplify fragments of a barley or cereal yellow dwarf virus, comprising:
one pair of primers including a first nucleic acid having the nucleotide sequence set forth in the Sequence Listing as SEQ ID NO: 1 and a second nucleic acid having the nucleotide sequence set forth in the Sequence Listing as SEQ ID NO: 2, and
the other pair of primers including a first nucleic acid having the nucleotide sequence set forth in the sequence listing as SEQ ID NO: 1 and a second nucleic acid having the nucleotide sequence set forth in the Sequence Listing as SEQ ID NOS: 3 or 4.

8. Oligonucleotide primers for a polymerase chain reaction to amplify fragments of Subgroup I and Subgroup II barley or cereal yellow dwarf viruses, comprising:
forward primers having the nucleotide sequences set forth in the Sequence Listing as SEQ ID NOS: 2, 5, and 3 or 4, and
reverse primers having the nucleotide sequences set forth in the Sequence Listing as SEQ ID NOS: 1 and 6 or 7.

9. Oligonucleotide primers for a polymerase chain reaction to amplify fragments of Subgroup I and Subgroup II barley and cereal yellow dwarf viruses, comprising forward primers having the nucleotide sequences set forth in SEQ ID NOS: 2, 8, and 3 or 4, and reverse primers having the nucleotide sequences set forth in SEQ ID NOS: 1 and 6 or 7.
